(19) United States
(12) Patent Application Publication
(10) Pub. No.: US 2013/0245560 A1
(43) Pub. Date: Sep. 19, 2013

(54) NASAL DEVICE

(71) Applicants: Miguel Ramón Guillem Garcia, VILLENA (ES); Carlos Antonio Guillem Garcia, VILLENA (ES); Alvaro Guillem Garcia, VILLENA (ES)

(72) Inventors: Miguel Ramón Guillem Garcia, VILLENA (ES); Carlos Antonio Guillem Garcia, VILLENA (ES); Alvaro Guillem Garcia, VILLENA (ES)

(21) Appl. No.: 13/554,352

(22) Filed: Jul. 20, 2012

(30) Foreign Application Priority Data
Mar. 15, 2012 (ES) ES 201230292U

(51) Int. Cl. A61M 1/00

(52) U.S. Cl. 604/190

(57) ABSTRACT

The invention consists of a nasal aspirator like a syringe, configured from a hollow tubular body with a nozzle that may or may not be interchangeable, that is provided with a filter and incorporates in the inner part a plunger formed by a front top, a central rod and a rear push element, having said push element holding means sized and configured for the user's thumb, whose holding means consist of a ring opened and formed by respective curved appendages, arising perpendicularly from the circular base defining said push element and being oriented such that they face one another.

### OBJECT OF THE INVENTION

As expressed in the title of the present specification, the invention relates to a nasal aspirator that provides several advantages and novel features to its intended function, which advantages and novel features involve an improvement with respect to the systems known today for the same purpose and will be described in detail below.

More particularly, the object of the invention focuses on a nasal hygiene device, preferably applicable for use in young children and babies which, being of the type that serves for aspirating mucus through aspirating same by means of a plastic syringe with a special nozzle for said use, has the particularity of having an innovative structural design especially envisaged to facilitate the use thereof with only one hand, making said task much easier and practical.

### FIELD OF APPLICATION OF THE INVENTION

The field of application of the present invention is encompassed within the technical sector of the industry dedicated to manufacturing hygiene apparatuses and devices, focusing particularly on the scope of syringe-like nasal aspirators and more specifically on those intended for infant use.

### BACKGROUND OF THE INVENTION

Achieving proper nasal hygiene in babies, who are incapable of blowing their own noses, and yet keeping their upper respiratory tracts clear is very important and necessary especially when they have a cold; many methods are known the most common being aspirating mucus by means of cannulas, by means of rubber bulbs or by means of syringes, the latter being the safest and the most practical system and it is the system taken into consideration by the present invention, since concerning the first two systems, in the case of bulb, it has become obsolete due to it being unhygienic, and in the case of a cannula, in which the mother performs the extraction by means of aspirating, it is also rather unhygienic.

On the other hand, in the third case, i.e., by means of syringes, all of them have the drawback that, to carry them out correctly and to achieve a good suction of mucus, it is necessary to use both hands, which means that the baby cannot be held properly at the same time, said nasal hygiene task being turned into a task which is difficult to be performed by a single person.

The objective of the present invention is to develop a new type of nasal aspirator syringe which can easily be handled by the mother or the babysitter with only one hand, thus allowing holding the baby's head with the other hand and performing the operation safely and assuring that the child, who is generally reluctant to allow one to introduce a foreign instrument into his/her nose, is not hurt.

On the other hand it must be highlighted that the applicant is unaware of the existence of any other nasal aspirator or invention of similar application having technical, structural and constitutive features similar to those presented by the claimed nasal aspirator proposed herein.

### SUMMARY OF THE INVENTION

Therefore, the nasal aspirator proposed by the present invention is configured as a remarkable novelty within its field of application, since the objective described above as ideal is specifically and satisfactorily achieved based on its implementation, the characterizing details thereof which make it possible and which distinguish it from that already known on the market being conveniently listed in the final claims accompanying the present specification of same.

Specifically, as has been discussed above, the invention proposes a nasal aspirator, particularly a nasal aspirator for babies of the type which is configured like a plastic syringe formed by a tubular body and a suction plunger, to which there is coupled a rounded nozzle especially designed to be able to be introduced into the nostril of children without hurting them.

Based on said already known conventional configuration, the proposed aspirator has the particularity of having in the rear part of the plunger, which is the part which, by means of its movement, creates the vacuum inside the body of the syringe and, therefore, causes the suction of the mucus when the nozzle is inserted into the nose of the baby, said plunger has, in said rear part, a ring-shaped handle or termination which is especially sized and configured to allow introducing therein the thumb of the person performing the nasal hygiene, said thumb being of the very hand that is holding the body of the syringe.

Therefore with only one hand, said person performing the nasal hygiene with the aspirator of the invention can hold the body of the syringe and move the plunger backwards to aspirate the mucus, making the use of both hands unnecessary and one hand is enough, whereas he/she could hold the child's head with the other hand.

In view of the foregoing, it is stated that the described nasal aspirator represents an innovative structure of features unknown until now for such purpose, these reasons and its practical use providing it with sufficient foundation to obtain the privilege of exclusivity sought.

### DESCRIPTION OF THE DRAWINGS

To complement the description that is being made for the aspirator of the invention and for the purpose of aiding to better understand the features distinguishing it, a set of drawings is attached to the present specification as an integral part thereof in which the following has been depicted with an illustrative and non-limiting character:

FIG. 1 shows an elevational cross-section view of an embodiment of the nasal aspirator object of the invention, the parts and elements which it comprises, as well the configuration and arrangement thereof being seen therein.

FIG. 2 shows a perspective view of the way of using the nasal aspirator example according to the invention, shown in the preceding figure, which has been depicted with the plunger in its resting position.

FIG. 3 shows a view similar to the preceding figure of the nasal aspirator of the invention, depicting, in this case, the plunger in its suction position.

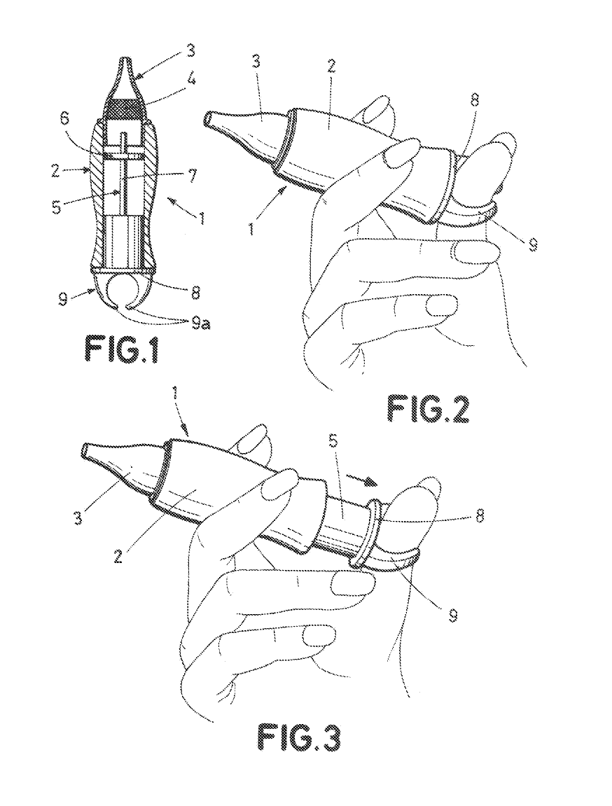

### PREFERRED EMBODIMENT OF THE INVENTION

In view of the mentioned drawings and according to the numbering used, a preferred embodiment of the invention can be seen therein, which comprises the parts and elements described in detail below.

Therefore, as observed in said drawings, the nasal aspirator (1) in question is configured, like a syringe, from a hollow tubular body (2) which incorporates in the front part a nozzle (3) which can preferably be interchanged and is internally provided with a filter (4), and incorporates in the inner part a plunger (5) formed by a front stop (6), a central rod (7) and a rear push element (8).

Said plunger (5), the movement of which from a resting position in which it is introduced into the hollow tubular body (2) to a working position in which it moves towards the rear part of said tubular body (2), determines the intended aspirating effect through said nozzle (3), has the particularity of having, in said rear push element (8), a ring (9) sized and configured such that it determines holding means to allow introducing therein the user's thumb, whereby the plunger (5) could be moved backwards, as shown in FIG. 3, while at the same time holding the body (2) with the same hand.

It must be highlighted that said ring (9) of the rear push element (8) of the plunger (5) is preferably an open ring which is formed by respective curved appendages (9a) arising perpendicularly from the circular base defining said push element (8), being oriented such that they face one another to define the mentioned ring (9) without closing it. Certain elasticity is thereby achieved, allowing adapting it to the different user finger sizes.

Having sufficiently described the nature of the present invention as well as the manner of carrying it out to practice, it is not considered necessary to further describe the invention so that any person skilled in the art can comprehend the scope thereof and advantages derived from it, stating that it could be carried out to practice within its essentiality in other embodiments differing in detail from that indicated by way of example, which embodiments will also achieve the protection sought provided that the fundamental principle thereof is not altered, changed or modified.

1. A nasal aspirator of the type which, like a syringe, is configured from a hollow tubular body (2) which incorporates in the front part a nozzle (3) that may or may not be interchangeable, and internally provided with a filter (4), and incorporates in the inner part a plunger (5) formed by a front stop (6), a central rod (7) and a rear push element (8), characterized in that said plunger (5) has holding means sized and configured for the user's thumb in its rear push element (8).

2. The nasal aspirator according to claim 1, characterized in that said holding means for the user's thumb in the rear push element (8) of the plunger (5) consist of a ring (9).

3. The nasal aspirator according to claim 2, characterized in that the ring (9) of the rear push element (8) of the plunger (5) is open and formed by respective curved appendages (9a) arising perpendicularly from the circular base defining said push element (8), being oriented such that they face one another.
